(19) United States
(12) Patent Application Publication
(10) Pub. No.: US 2017/0368083 A1
(43) Pub. Date: Dec. 28, 2017

(54) GINSENOSIDE COMPOSITION, AND PREPARATION METHOD AND USE THEREOF

(71) Applicant: INFINITUS (CHINA) COMPANY LTD., Jiang Men City (CN)

(72) Inventors: Qingtao YU, Jiang Men City (CN); Yazhong GE, Jiang Men City (CN); Chung Wah MA, Jiang Men City (CN); Chuixin QIN, Jiang Men City (CN)

(73) Assignee: INFINITUS (CHINA) COMPANY LTD., Jiang Men City (CN)

(21) Appl. No.: 15/388,094

(22) Filed: Dec. 22, 2016

(30) Foreign Application Priority Data
Jun. 24, 2016 (CN) 201610485809.7
Aug. 16, 2016 (CN) 201610674888.6

(51) Int. Cl. A61K 31/704

(52) U.S. Cl. CPC A61K 31/704

(57) ABSTRACT

The present invention relates to the pharmaceutical field, and in particular to a ginsenoside composition, and preparation method and use thereof. The ginsenoside composition comprises rare ginsenosides Rh4, Rk3, Rk1 and Rg5. By conducting a systematic research on the change in active ingredients during the steaming process of ginseng and applying response surface to design experiments so as to optimize the ginseng processing process, a processing method wherein there is the most significant change in ginsenoside ingredients relative to the original medicinal material, and especially a processing method for efficiently obtaining rare ginsenoside ingredients, is unexpectedly discovered in the present invention. Through later screening of pharmacological activities, the inventors further unexpectedly discovered that the processed ginseng product rich in rare ginsenosides has a significant efficacy in invigorating kidney yang.

### CROSS REFERENCE OF RELATED APPLICATION

This application claims the benefit of priorities to Chinese Patent Application No. 201610674888.6 titled “GINSENOSIDE COMPOSITION, AND PREPARATION METHOD AND USE THEREOF”, filed with the Chinese State Intellectual Property Office on Aug. 16, 2016, and Chinese Patent Application No. 201610485809.7 titled “GINSENOSIDE COMPOSITION AND METHOD OF PREPARING THE SAME”, filed with the Chinese State Intellectual Property Office on Jun. 24, 2016, the entire disclosures of which are incorporated herein by reference.

### FIELD

The present invention relates to the pharmaceutical field, and in particular to a ginsenoside composition, and preparation method and use thereof.

### BACKGROUND

Ginseng is dry root and rhizome of Panax ginseng C. A. Mey. It is warm, tastes sweet, and has an efficacy of invigorating primodial qi, invigorating spleen for benefiting lung and promoting fluid production for nourishing blood. Modern researches showed that ginseng plays a significant role in enhancing body immunity, improving myocardial ischemia and the like.

The processing history of ginseng is originated from the Han dynasty. Red ginseng is the most common processed product of ginseng, and is prepared by processing processes comprising screening the fresh ginseng, suncuring, cleaning, steaming, and oven-drying. Ginsenosides are primary effective active ingredients in ginseng and various processed products thereof.

During the steaming process of ginseng, both the species and contents of ginsenosides therein would vary as the steaming duration, temperature and times change. However, such changes are uncertain, meaning that the change in ginsenoside species and contents caused by the change of steaming parameters completely cannot be expected accurately.

Therefore, it is of important practical significance to provide a ginsenoside composition, and a preparation method thereof

### SUMMARY

In view of the above, the present invention provides a ginsenoside composition, and preparation method and use thereof. An object of the present invention is to achieve effective extraction of ginsenosides, solve the defect in the prior art of difficulty in obtaining rare ginsenosides, and provide an efficient, stable and high value-added extraction process. Meanwhile, the present invention further provides a product with a significant efficacy in invigorating kidney yang.

In order to achieve the above object, the following technical solutions are provided.

The present invention provides a ginsenoside composition comprising rare ginsenosides Rh4, Rk3, Rk1 and Rg5.

In some particular embodiments, the ginsenoside composition provided by the present invention further comprises ginsenosides Rf, Rb1, Rc, Rb2 and Rd.

In some particular embodiments, the ginsenoside composition provided by the present invention further comprises ginsenosides Rg1 and Re.

In some particular embodiments, in the ginsenoside composition provided by the present invention, the total content of the rare ginsenosides Rh4, Rk3, Rk1 and Rg5 is greater than 42.55 mg/g; the total content of the ginsenosides Rf, Rb1, Rc, Rb2 and Rd is less than 10 mg/g; and the total content of the ginsenosides Rg1 and Re is less than 0.35 mg/g.

In some particular embodiments, a method for preparing the ginsenoside composition provided by the present invention comprises steaming ginseng for 1 to 10 h at 90 to 130° C., wherein the steaming is carried out for at least 5 times.

In some particular embodiments, in the method for preparing the ginsenoside composition provided by the present invention, the steaming temperature is 100 to 120° C., preferably 110° C.

In some particular embodiments, in the preparation method for the ginsenoside composition provided by the present invention, the steaming is carried out for 1 to 9 times, preferably 1, 5 or 9 times.

In some particular embodiments, in the method for preparing the ginsenoside composition provided by the present invention, the steaming duration is 2 to 6 h, preferably 4 h.

The method for preparing the ginsenoside composition provided by the present invention further comprises pulverizing the steamed ginseng and passing the pulverized ginseng through a 100-mesh screen; or cutting the steamed ginseng into slices and extracting the slices with water or ethanol to obtain an extract.

In some particular embodiments of the present invention, the extraction is carried out twice with 75% alcohol, and each extraction is carried out for 4 h; after suction filtration, the dregs are extracted once under hot reflux with 200 ml of water, and the alcohol extract is evaporated to dryness under reduced pressure and mixed with the aqueous extract. The administration dosage of the aqueous extract is converted according to the original administration dosage. Thereafter, the aqueous extract is concentrated to a concentration for administration.

The present invention further provides use of the ginsenoside composition in the preparation of a medicament and/or healthcare product for increasing thymus or spleen index, plasma SOD activity and/or plasma cAMP/cGMP level.

The present invention further provides use of the ginsenoside composition in the preparation of a medicament and/or healthcare product for treating deficiency of kidney yang.

In some particular embodiments of the present invention, the medicament is administrated at a dosage of 0.2 to 2 g/kg/d for a non-human animal subject, and at a human equivalent dosage of 0.02 to 0.2 g/kg/d for a human subject.

The present invention provides a method for preparing the ginsenoside composition, wherein ginseng is steamed for 1 to 10 h at 90 to 130° C., wherein the steaming is carried out for at least 5 times.

In some particular embodiments, the steaming temperature in the preparation method provided by the present invention is 100 to 120° C., preferably 110° C.

In some particular embodiments, the steaming in the preparation method provided by the present invention is carried out for 1 to 9 times, preferably 1, 5 or 9 times.

In some particular embodiments, the steaming duration in the preparation method provided by the present invention is 2 to 6 h, preferably 4 h.

In some particular embodiments, the ginsenoside composition provided by the present invention is prepared as follows: steaming ginseng at 120° C. for 4 h and repeating the steaming for 5 more times to prepare the ginsenoside composition.

In some other particular embodiments, the ginsenoside composition provided by the present invention is prepared as follows: steaming ginseng at 120° C. for 4 h and repeating the steaming for 5 more times; then pulverizing and passing through a 100-mesh screen to obtain fine powders, extracting the fine powders twice with 75% alcohol, and each extraction is carried out for 4 h; after suction filtration, extracting the dregs once under hot reflux with 200 ml of water, evaporating the alcohol extract to dryness under reduced pressure and mixing it with the aqueous extract.

By conducting a systematic research on the change in active ingredients during the steaming process of ginseng and applying response surface to design experiments so as to optimize the ginseng processing process, a processing method wherein there is the most significant change in ginsenoside ingredients relative to the original medicinal material, and especially a processing method for efficiently obtaining rare ginsenoside ingredients, is unexpectedly discovered in the present invention.

As is shown by the experimental results, if the processing times keep constant, the total ginsenoside content is increased under the same processing duration when the temperature increases from 100 to 120° C.; if the processing duration keeps constant, the total ginsenoside content is increased under the same processing times when the temperature increases from 100 to 120° C. The results indicate that temperature is the most important factor, and processing times and duration have an interaction effect on the ginsenoside content.

During the steaming of ginseng, the species of ginsenosides therein vary depending on the difference in the processing temperature, duration and times. Ginsenosides Rg1 and Re with a large polarity have a gradually decreased content and even disappear, with increase of processing temperature, increase of processing times and extension of processing duration. Ginsenosides Rf, Rb1, Rc, Rb2 and Rd with a moderate polarity are relatively stable. However, rare ginsenosides RK3, Rh4, Rk1 and Rg5 are most affected by the processing process, with an obviously changed content. The results indicate that, there is a maximum change rate in the rare ginsenoside content under a processing process of 120° C., 4 h and 5 times; the more times the steaming is carried out for, the more significantly the rare ginsenoside content changes. Meanwhile, the rationality of nine-steam-nine-bask processing is verified.

Through later screening of pharmacological activities, the inventors further unexpectedly discovered that the processed ginseng product rich in rare ginsenosides has a significant efficacy in invigorating kidney yang.

The experimental results indicate that the thymus and spleen indexes are the best in R3 group; P <0.01 for cAMP/cGMP between the administration and model groups. It is shown that, the R3 aqueous extract indeed has a certain efficacy. The SOD result shows that antioxidant ability of the body is improved in both the positive medicament and R3 groups. In view of the above, the ginseng processed product R3 effectively improves the body temperature, thymus index and spleen index of the model mouse with deficiency of kidney yang. In addition, as compared to the model group, the ginseng processed product R3 obviously increases the SOD activity and also significantly increases the plasma cAMP/cGMP level. It is suggested that the ginseng processed product R3 improve the body function of mouse with deficiency of kidney yang.

### BRIEF DESCRIPTION OF THE DRAWINGS

In order to illustrate examples of the present invention or technical solutions in the prior art more clearly, drawings required to be used in the description of the examples or prior art will be introduced briefly below.

FIG. 1 shows a response surface plot of interaction factors of the processing duration and times versus ginsenoside Rb1;

FIG. 2 shows a response surface plot of interaction factors of the processing duration and times versus ginsenoside Rb1;

FIG. 3 shows a response surface plot of interaction factors of the processing duration and times versus ginsenoside Rb1;

FIG. 4 shows a plot comparing the content change of ginsenosides with different polarities;

FIG. 5 shows a schematic diagram of the cracking process of ginsenosides;

FIG. 6 shows a plot comparing the thymus index of mice;

FIG. 7 shows a plot comparing the spleen index of mice;

FIG. 8 shows a plot of the total plasma SOD level in mice;

FIG. 9 shows a plot comparing the body temperature of mice;

FIG. 10 shows a plot comparing the body temperature of four groups of mice on day 10 of the modeling;

FIG. 11 shows a plot comparing the thymus index of four groups of mice;

FIG. 12 shows a plot comparing the spleen index of four groups of mice;

FIG. 13 shows a plot of the plasma cAMP/cGMP level in mice;

FIG. 14 shows a plot assaying the plasma SOD activity in mice;

FIG. 15 shows a plot of the plasma testosterone level in mice;

In the drawings, C is the control group, M is the model group, G is the suncured ginseng group, R1 is the processed product group prepared in Example 1, R2 is the processed product group prepared in Example 2, R3 is the processed product group prepared in Example 3, R6 is the processed product group prepared in Example 6, and × is the positive medicament group.

### DETAILED DESCRIPTION

The present invention discloses a ginsenoside composition, and preparation method and use thereof, which can be achieved by those skilled in the art in light of the disclosure herein via appropriately improving process parameters. It is to be particularly specified that all similar substitutions and changes are apparent to those skilled in the art and deemed to be within the present invention. Although the method and use of the present invention have been described by means of preferred examples, it is apparent to relevant personnel that modifications or proper alterations and combinations can be made to the method and use described herein without departing from the content, spirit and scope of the present invention, so as to achieve and apply the technique of the present invention.

It is well known in the field of traditional Chinese medicine that during the steaming process of ginseng, both the species and content of ginsenosides therein would vary as the steaming duration, temperature and times change. To this end, in the present invention, a processing method wherein there is the most significant change in ginsenoside ingredients relative to the original medicinal material is determined in the process of attempting to optimize the ginseng processing process. On this basis, in the present invention, the content changes of ginsenosides in 20 different processed samples were analyzed by HPLC-MS and HPLC-UV. The results show that, three processing methods wherein the ginsenoside content and species change most significantly are: (1) 100° C., 2 h and 1 time; (2) 100° C., 6 h and 1 time; (3) 120° C., 4 h and 5 times, respectively. In the three methods, the species and content of ginsenoside ingredients are different from each other.

Meanwhile, it also has been unexpectedly discovered in the present invention that the ginseng obtained by high-frequency steaming has an increased content of rare ginsenosides, and especially an obviously increased content of ginsenosides RK3, Rh4, Rk1 and Rg5. Such a steamed product can significantly improve diseases associated with deficiency of kidney yang.

Therefore, in a first aspect, the present invention provides a ginsenoside composition for treating deficiency of kidney yang, comprising a rare ginsenoside as the main active ingredient.

In the first aspect, the present invention provides a ginsenoside composition for treating deficiency of kidney yang, wherein the rare ginsenoside comprises ginsenosides Rh4, Rk3, Rk1 and Rg5.

In the first aspect, the present invention provides a ginsenoside composition for treating deficiency of kidney yang, which further comprises a small amount of ginsenosides Rf, Rb1, Rc, Rb2 and Rd.

In the first aspect, the present invention provides a ginsenoside composition for treating deficiency of kidney yang, which may further comprise a small amount of ginsenosides Rg1 and Re.

In the first aspect, the present invention provides a ginsenoside composition for treating deficiency of kidney yang, comprising rare ginsenosides Rh4, Rk3, Rk1 and Rg5; ginsenosides Rf, Rb1, Rc, Rb2 and Rd; as well as optionally ginsenosides Rg1 and Re.

In the first aspect, the present invention provides a ginsenoside composition for treating deficiency of kidney yang, comprising rare ginsenosides Rh4, Rk3, Rk1 and Rg5 in a total content of larger than 42.55 mg/g; ginsenosides Rf, Rb1, Rc, Rb2 and Rd in a total content of less than 10 mg/g; as well as ginsenosides Rg1 and Re in a total content of less than 0.35 mg/g.

In the first aspect, the present invention provides a ginsenoside composition for treating deficiency of kidney yang, comprising rare ginsenosides Rh4, Rk3, Rk1 and Rg5 in a total content of 42.55 to 129.8 mg/g; ginsenosides Rf, Rb1, Rc, Rb2 and Rd in a total content of 1 to 10 mg/g; as well as ginsenosides Rg1 and Re in a total content of 0 to 0.35 mg/g.

In a second aspect, the present invention provides a steamed ginseng product, which has been subjected to high-frequency processing under a temperature of 90 to 130° C. for a duration of 1 to 10 h, wherein the processing is preferably performed for at least 5 times.

The steamed ginseng product according to the second aspect of the present invention, wherein the processing temperature is 100 to 120° C., preferably 100° C., preferably 110° C., and preferably 120° C.

The steamed ginseng product according to the second aspect of the present invention, wherein the processing duration is 2 to 6 h, preferably 2 h, preferably 4 h, and preferably 6 h.

The steamed ginseng product according to the second aspect of the present invention, wherein the processing is carried out for at least 5 times, preferably 5 times, and preferably 9 times.

The ginseng steamed product according to the second aspect of the present invention, wherein the processing temperature is 90 to 130° C. and the duration is 1 to 10 h, characterized in that the processing is performed for at least 5 times, preferably 5 times, and preferably 9 times.

In a third aspect, the present invention provides a method for preparing a steamed ginseng product, which is prepared according to the processing condition provided in the first aspect.

In a fourth aspect, the present invention provides use of a steamed ginseng product in the preparation of a medicament for treating deficiency of kidney yang, wherein the ginseng steamed product is as described in the first aspect.

Hereinafter, the present invention is further illustrated. The entire contents of the references cited in the present invention and references cited therein are incorporated herein by reference.

In the present invention, any technical feature of any technical solution in any aspect thereof is also applicable to any embodiment in any aspect thereof, as long as they would not contradict and such a mutual application could be appropriately modified when necessary.

All the raw materials and reagents used in the ginsenoside composition, and preparation method and use thereof provided in the present invention are commercially available.

Hereinafter, the present invention is further explained in conjunction with examples.

### EXAMPLE 1

### Processing of a Ginsenoside Composition

Ginseng was steamed at 100° C. for 2 h. The steaming was repeated once more to prepare the ginsenoside composition.

### EXAMPLE 2

### Processing of a Ginsenoside Composition

Ginseng was steamed at 100° C. for 6 h. The steaming was repeated once more to prepare the ginsenoside composition.

### EXAMPLE 3

### Processing of a Ginsenoside Composition

Ginseng was steamed at 120° C. for 4 h. The steaming was repeated 5 more times to prepare the ginsenoside composition.

### EXAMPLE 4

### Processing of a Ginsenoside Composition

Ginseng was steamed at 90° C. for 10 h. The steaming was repeated 9 more times to prepare the ginsenoside composition.

### EXAMPLE 5

### Processing of a Ginsenoside Composition

Ginseng was steamed at 130° C. for 1 h. The steaming was repeated 4 more times to prepare the ginsenoside composition.

### EXAMPLE 6

### Processing of a Ginsenoside Composition

The ginseng prepared in Example 3 was pulverized and passed through a 100-mesh screen to obtain fine powders, which were extracted twice with 75% alcohol and each extraction was carried out for 4 h. After suction filtration, the dregs were extracted under hot reflux once with 200 ml water. The alcohol extract was evaporated to dryness under reduced pressure and mixed with the aqueous extract.

### EXAMPLE 7

### Effect of the Processing Process on Ginsenosides in Ginseng

### 1.1 Analysis of Ginsenoside Ingredients

Original medicinal materials of suncured ginseng from the same patch were taken, from which 20 normal processed products were prepared using a response surface method (see Table 7). The effect of the processing means on the chemical composition of the ginseng processed products was investigated. With HPLC-MS and HPLC-UV methods, the change in the ginsenoside species and content was tracked dynamically, ingredients of newly generated ginsenosides were identified, and the change rule of ginsenosides was revealed.

### 1.1.1 Formulation of Test Solutions

The dry original medicinal materials of suncured ginseng and dry processed products were pulverized into powders and passed through an 80-mesh screen, respectively. 2 g of each of the dry original medicinal materials of suncured ginseng and powders of dry processed products prepared in Examples 1 to 3 was accurately weighed (as compared to the original medicinal materials, the mass of the processed product was substantially unchanged after oven-drying), and put in a 100 ml Erlenmeyer flask with cover. 40 ml of 70% ethanol was added and the mixture was sonicated in an ice-water bath for 60 min (100 Hz). The extract was filtered using an ordinary filter paper, concentrated under reduced pressure at 45° C. using a rotory evaporator to remove ethanol to near-dryness, and redissolved to 6 ml with 70% ethanol. The mixture was centrifuged at 13000 rpm for 10 min. The supernatant was taken for chromatographic analysis.

### 1.1.2 Preparation of Control Solutions

13 ginseng standards were used in the present experiment, which could be divided into two types based on their structures: protopanoxadiol (PPD) type, including ginsenosides Rb1, Rc, Rb2, Rd, 20(R)-Rg3, 20(S)-Rg3, Rk1 and Rg5; and Rg1, Re, Rf, Rk3 and Rh4 of protopanaxatriol (PPT) type. 1.00 mg of each of the 13 kinds of ginsenoside controls was accurately weighed, dissolved in a 1 ml volumetric flask and formulated into a 1 mg/ml of mixed standard solution.

### 1.1.3 Conditions for Chromatographic Analysis

Agilent 1260 High Performance Liquid System (produced by Agilent Technologies Inc., USA) comprising an on-line vacuum degasser, high pressure quaternary pump, disc autosampler and column oven was used for chromatographic analysis. Agilent Zorbax Extend-C18 column (4.6 x 250 mm, 5μm) was used for chromatographic separation with pure water as mobile phase A and acetonitrile as mobile phase B. The gradient elution condition was 0 to 13 min, 19% to 21% B; 13 to 17 min, 21% to 26% B; 17 to 20 min, 26% B; 20 to 24 min, 26% to 30% B; 24 to 37 min, 30% to 45% B; 37 to 46 min, 45% to 49% B; 46 to 51 min, 49% to 52% B; 51 to 54 min, 52% to 53% B; 54 to 56 min, 53% to 55% B. Flow rate: 1 ml/min; wavelength: 203 nm; column temperature: 25° C.; loading volume: 20 pl.

### 1.1.4 Conditions for Mass Spectrometry

A liquid phase condition was used. Post-column flow splitting was performed and 50% eluent entered into Q-TOF-MS for detection. Agilent 6520 Q-TOF-MS system (produced by Agilent Technologies Inc., USA) with an electrospray (ESI) ion source was used as the detector. Detection conditions were: dry gas (N2) flow rate: 10 1/min; dry gas temperature: 320° C.; atomizer pressure: 35 psig; capillary voltage: 3500 V; OCT RFV: 750 V; fragment voltage: 120 V. Data were acquired in a negative ion mode with an ion scanning range of m/z 100 to 1500. The secondary collision energy (CE) was set to 15 to 70 V and the loading volume was 1 μl.

Agilent Mass Hunter Acquisition Software Version B.04.00 and Mass Hunter Workstation Software Version B.05.00 software (Agilent Technologies Inc.) were used for data acquisition and processing, respectively.

### 2. Experimental Results and Discussion

### 2.1 Analysis Results of Ginsenoside Ingredients

According to the difference in the processing temperature, duration and times, the processing temperature under investigation included 100° C., 110° C. and 120° C.; the processing duration included 2 h, 4 h and 6 h; and the processing times included 1 time, 5 times and 9 times. A response surface analysis method with 3 factors and 3 levels was designed using Box-Behnken central composite experimental design principle to generate 20 groups of processing processes. The effect of different steaming processes on the ginseng active ingredient in suncured ginseng from the same patch was compared. The research results indicated that the processing process resulted in a decrease in the content of some chemical ingredients. Therefore, the processing process significantly changed the ginsenoside ingredients of suncured ginseng.

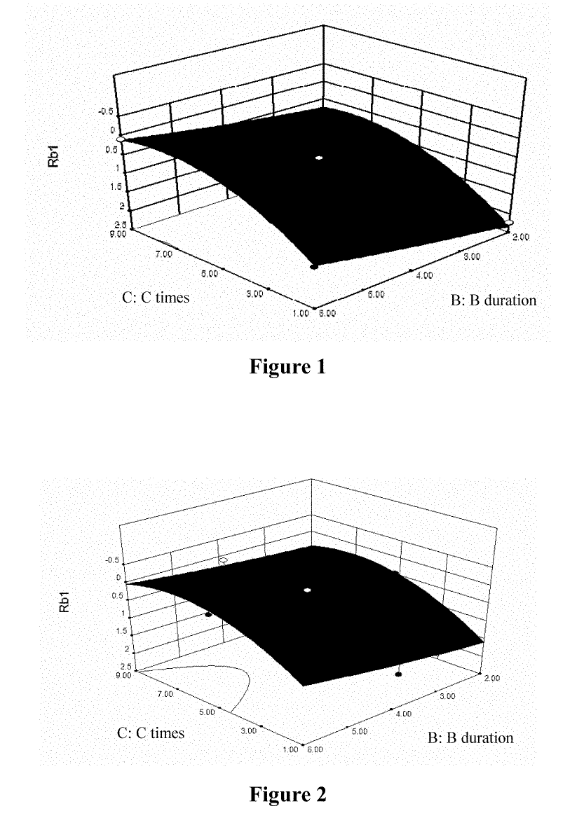

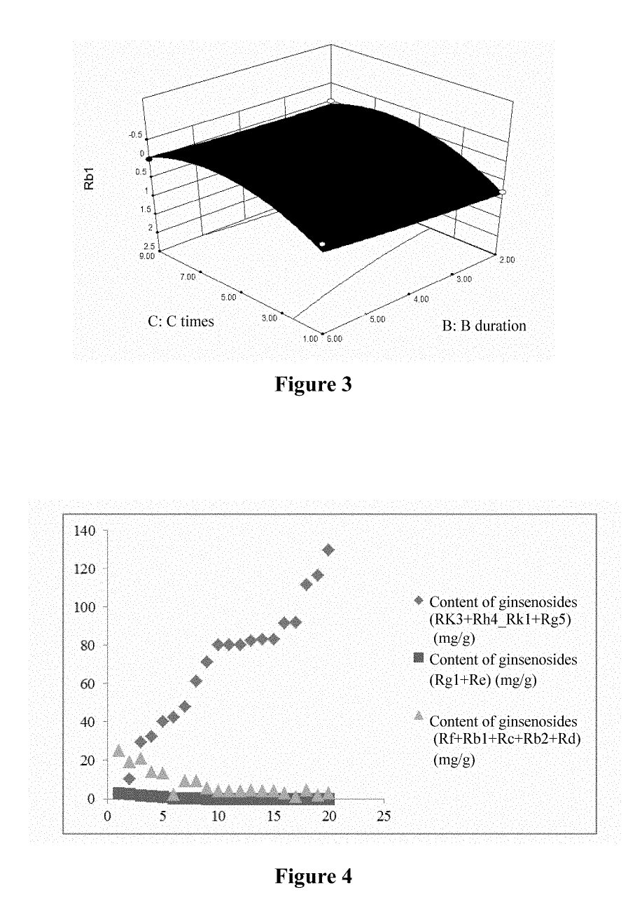

Taking a single ginsenoside Rb1 as a representative, the chemical composition of the processed ginseng products was analyzed. The results were shown in FIGS. 1, 2 and 3, which represented response surface plots of interaction factors of the processing duration and times versus ginsenoside Rb1.

The chemical composition of the processed ginseng product was analyzed with the total ginsenoside content as a representative detection index. As shown in Tables 1, 2, 3, 4, 5 and 6, when the processing times kept constant, the total ginsenoside content was increased under the same processing duration when the temperature increased from 100° C. to 120° C. As shown in Tables 4, 5 and 6, when the processing duration kept constant, the total ginsenoside content was increased under the same processing times when the temperature increased from 100° C. to 120° C. The results indicated that the temperature is the most important factor among the three factors, and the processing times and duration had an interaction effect on the ginsenoside content.

TABLE 1
Comparison table for different processing temperatures
and durations when processing was carried out once

| Processing | Processing | Processing | Total ginsenoside |
|---|---|---|---|
| temperature | duration | times | content (mg/g) |
| 100° C. | 2 h | 1 | 30.0775 |
| 100° C. | 6 h | 1 | 32.021 |
| 120° C. | 2 h | 1 | 47.2815 |
| 120° C. | 6 h | 1 | 76.8015 |
| 110° C. | 4 h | 1 | 52.1035 |

TABLE 2
Comparison table for different processing temperatures and
durations when processing was carried out for 5 times

| Processing | Processing | Processing | Total ginsenoside |
|---|---|---|---|
| temperature | duration | times | content (mg/g) |
| 100° C. | 4 h | 5 | 57.4650 |
| 110° C. | 2 h | 5 | 70.8705 |
| 100° C. | 6 h | 5 | 115.7535 |
| 120° C. | 4 h | 5 | 132.4870 |
| 110° C. | 4 h | 5 | 83.9730 |
| 110° C. | 4 h | 5 | 84.0215 |
| 110° C. | 4 h | 5 | 84.1415 |
| 110° C. | 4 h | 5 | 87.2540 |
| 110° C. | 4 h | 5 | 86.3705 |
| 110° C. | 4 h | 5 | 86.8680 |

TABLE 3
Comparison table for different processing temperatures and
durations when processing was carried out for 9 times

| Processing | Processing | Processing | Total ginsenoside |
|---|---|---|---|
| temperature | duration | times | content (mg/g) |
| 100° C. | 2 h | 9 | 54.4050 |
| 100° C. | 6 h | 9 | 44.7260 |
| 120° C. | 2 h | 9 | 118.1585 |
| 120° C. | 6 h | 9 | 92.8790 |
| 110° C. | 4 h | 9 | 94.6395 |

TABLE 4
Comparison table for different processing temperatures
and times when processing was carried out for 2 h

| Processing | Processing | Processing | Total ginsenoside |
|---|---|---|---|
| temperature | duration | times | content (mg/g) |
| 100° C. | 2 h | 1 | 30.0775 |
| 100° C. | 2 h | 9 | 54.4050 |
| 120° C. | 2 h | 1 | 47.2815 |
| 120° C. | 2 h | 9 | 118.1585 |
| 110° C. | 2 h | 5 | 70.8705 |

TABLE 5
Comparison table for different processing temperatures
and times when processing was carried out for 6 h

| Processing | Processing | Processing | Total ginsenoside |
|---|---|---|---|
| temperature | duration | times | content (mg/g) |
| 100° C. | 6 h | 1 | 32.0210 |
| 100° C. | 6 h | 9 | 44.7260 |
| 120° C. | 6 h | 1 | 76.8015 |
| 120° C. | 6 h | 9 | 92.8790 |
| 100° C. | 6 h | 5 | 115.7535 |

TABLE 6
Comparison table for different processing temperatures
and times when processing was carried out for 4 h

| Processing | Processing | Processing | Total ginsenoside |
|---|---|---|---|
| temperature | duration | times | content (mg/g) |
| 100° C. | 4 h | 5 | 57.4650 |
| 120° C. | 4 h | 5 | 132.4870 |
| 110° C. | 4 h | 1 | 52.1035 |
| 110° C. | 4 h | 9 | 94.6395 |
| 110° C. | 4 h | 5 | 83.9730 |
| 110° C. | 4 h | 5 | 84.0215 |
| 110° C. | 4 h | 5 | 84.1415 |
| 110° C. | 4 h | 5 | 87.2540 |
| 110° C. | 4 h | 5 | 86.3705 |
| 110° C. | 4 h | 5 | 86.8680 |

During the steaming of ginseng, the species of ginsenosides therein vary depending on the difference in the processing temperature, duration and times. For the same processed product, ginsenosides therein were divided into 3 groups based on their polarity. As shown in Table 7, the change in the content of ginsenosides with different polarities was compared. As shown in FIG. 4, ginsenosides Rg1 and Re with a large polarity had a gradually decreased content and even disappear, with increase of processing temperature, increase of processing times and extension of processing duration. Ginsenosides Rf, Rb1, Rc, Rb2 and Rd with a moderate polarity were relatively stable. However, rare ginsenosides RK3, Rh4, Rk1 and Rg5 were most affected by the processing process, with an obviously changed content.

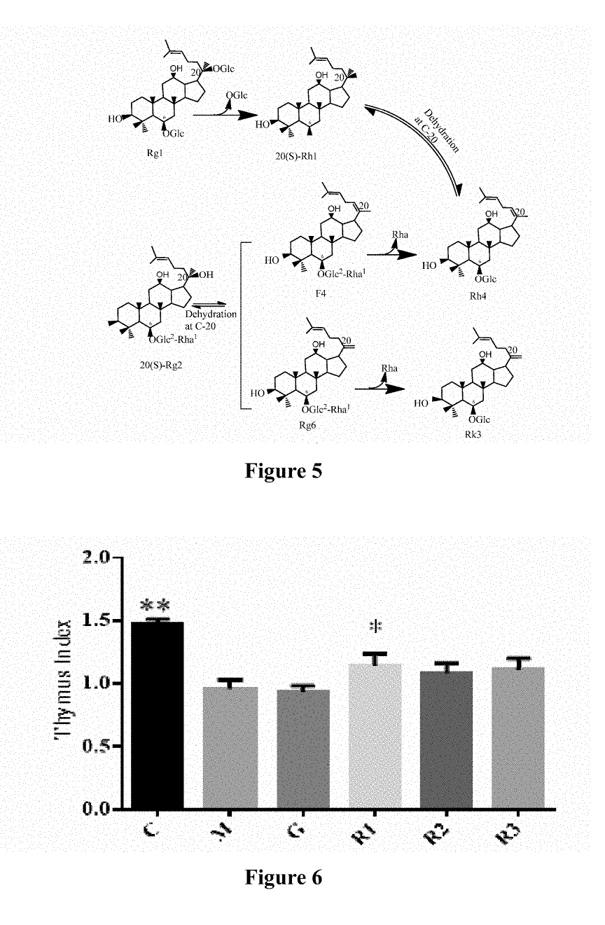

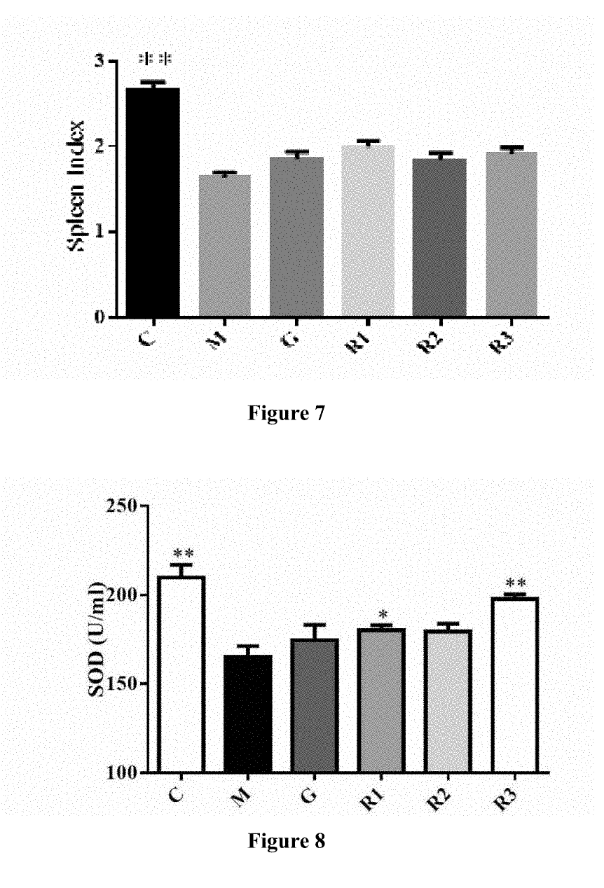

The original medicinal materials and processed products of suncured ginseng were analyzed quantitatively by HPLC-UV, and three processing means with the largest impact on the composition difference were finally screened, which were in sequence: (1) 100° C., 2 h and 1 time (FIG. 5); (2) 100° C., 6 h and 1 time (FIG. 6); (3) 120° C., 4 h and 5 times (FIG. 7). These three processing means were selected for the following reasons: in the processing process (1), ginsenosides Rg1 and Re with a larger polarity have a higher content, and meanwhile relative to the original medicinal materials of suncured ginseng, a lower content of rare ginsenosides RK3, Rh4, Rk1, Rg5 and the like with a smaller polarity began to occur. In the processing process (2), ginsenosides Rg1 and Re with a large polarity are partially transformed into new rare ginsenosides Rh4 and the like with a small polarity by stepwise cracking; ginsenosides Rb1, Rc, Rb2 and Rd with a large polarity were deglycosylated and dehydrated step by step and finally Rg5, Rk1 and the like were generated. This was a transition of the transformation to ginsenosides with a smaller polarity, and all of the 13 kinds of ginsenosides under research were comprised. In the processing process (3), ginsenosides Rg1, Re, Rb1 and Rc with a larger polarity gradually disappeared and the newly generated rare ginsenosides Rh4, Rk3, Rk1 and Rg5 had a highest relative content. In theory, the change in the species and content of active ingredients in processed products would directly affect the pharmacological activity of ginseng. Therefore, the three processed products were studied in a subsequent activity study.

The results also indicated that, there was a maximum change rate in the rare ginsenoside content under the processing process of 120° C., 4 h and 5 times; the more times the steaming was carried out for, the more significantly the rare ginsenoside content changed. Meanwhile, the rationality of nine-steam-nine-bask processing was verified.

TABLE 7
Comparison table for the content change of ginsenosides with different polarities

| | | | | | Content of | |
|---|---|---|---|---|---|---|
| | Factor 1 | Factor 2 | | Content of | ginsenosides | Content of |
| | A: | B: | Factor 3 | ginsenosides | (Rf + Rb1 + Rc + | ginsenosides |
| | temperature | duration | C: times | (Rg1 + Re) | Rb2 + Rd) | (RK3 + Rh4_Rk1 + |
| Group | ° C. | h | times | (mg/g) | (mg/g) | Rg5) (mg/g) |
| 1 | 100 | 2 | 1 | 2.75 | 25.00 | 2.30 |
| 3 | 100 | 6 | 1 | 2.50 | 19.20 | 10.30 |
| 13 | 110 | 4 | 1 | 1.80 | 20.85 | 29.45 |
| 2 | 120 | 2 | 1 | 1.15 | 13.85 | 32.30 |
| 4 | 120 | 6 | 1 | 1.05 | 13.10 | 40.25 |
| 9 | 100 | 4 | 5 | 0.35 | 2.20 | 42.55 |
| 11 | 110 | 2 | 5 | 0.35 | 9.20 | 47.95 |
| 12 | 110 | 6 | 5 | 0.25 | 9.25 | 61.40 |
| 10 | 120 | 4 | 5 | 0.00 | 5.40 | 71.40 |
| 19 | 110 | 4 | 5 | 0.00 | 3.85 | 80.10 |
| 17 | 110 | 4 | 5 | 0.00 | 3.80 | 80.25 |
| 15 | 110 | 4 | 5 | 0.00 | 3.85 | 80.30 |
| 18 | 110 | 4 | 5 | 0.00 | 4.10 | 82.25 |
| 16 | 110 | 4 | 5 | 0.00 | 3.90 | 83.00 |
| 20 | 110 | 4 | 5 | 0.00 | 4.00 | 83.30 |
| 5 | 100 | 2 | 9 | 0.00 | 2.95 | 91.70 |
| 7 | 100 | 6 | 9 | 0.00 | 1.00 | 91.90 |
| 14 | 110 | 4 | 9 | 0.00 | 4.20 | 111.55 |
| 6 | 120 | 2 | 9 | 0.00 | 1.70 | 116.50 |
| 8 | 120 | 6 | 9 | 0.00 | 2.70 | 129.80 |

As compared to the original medicinal materials of suncured ginseng, the ginsenoside species and content changed after steaming. It is verified upon research that, in the steaming process, the structure of the original ginsenosides in suncured ginseng was unstable, deglycosylation and dehydration reactions mainly occurred and ginsenosides with smaller polarities were generated, wherein rare ginsenoside 20S/20R-Rg3 was a representative hydrolysis product, while Rk3/Rh4 and Rk1/Rg5 were products of deglycosylation followed by further dehydration at C-20, as could be seen from the cracking process shown in FIG. 5.

The ginsenoside compositions prepared in Examples 4-6 were tested as above. The experimental results were similar to the above test results without any significant difference (P>0.05).

### Example 8

### Effect of the Processing Process on the Activity of Strengthening Yang of Ginseng

### 1. Experiment Method

Mice were divided into the following 6 groups, each of which comprised 12 animals.

(1) Control group: 0.4 ml pure water was intragastrically administered daily for 10 consecutive days.

(2) Model group: 0.5 mg hydrocortisone was intragastrically administered daily for 10 consecutive days.

(3) Suncured ginseng group: suncured ginseng was pulverized and passed through a 100-mesh screen. The intragastrical administration was initiated 3 days prior to start of the experiment and lasted for 13 consecutive days. The administration dosage was 1 g/kg raw medicinal materials/d. Meanwhile, 0.5 mg/d of the molding agent hydrocortisone was administered for 10 consecutive days after 3 days of the administration.

(4) The processed product R1 group prepared in Example 1 (suncured ginseng was steamed once for 2 h at 100° C. as shown in Table 7). It was pulverized and passed through a 100-mesh screen. The intragastrical administration was initiated 3 days prior to start of the experiment and lasted for 13 consecutive days. The administration dosage was 1 g/kg raw medicinal materials/d. Meanwhile, 0.5 mg/d of the molding agent hydrocortisone was administered for 10 consecutive days after 3 days of the administration.

(5) The processed product R2 group prepared in Example 2 (suncured ginseng was steamed once for 6 h at 100° C. as shown in Table 7). It was pulverized and passed through a 100-mesh screen. The intragastrical administration was initiated 3 days prior to start of the experiment and lasted for 13 consecutive days. The administration dosage was 1 g/kg raw medicinal materials/d. Meanwhile, 0.5 mg/d of the molding agent hydrocortisone was administered for 10 consecutive days after 3 days of the administration. (6) The processed product R3 group prepared in Example 3 (suncured ginseng was steamed for 5 times for 4 h at 120° C. as shown in Table 7). It was pulverized and passed through a 100-mesh screen. The intragastrical administration was initiated 3 days prior to start of the experiment and lasted for 13 consecutive days. The administration dosage was 1 g/kg raw medicinal materials/d. Meanwhile, 0.5 mg/d of the molding agent hydrocortisone was administered for 10 consecutive days after 3 days of the administration.

On day 11, the experiment animals were bled to collect the whole blood by eyeball enucleation and sacrificed. Various organs were harvested. The spleen index, thymus index and plasma SOD level of the experimental animals were detected and averaged, so as to screen a processing process which had the best effect on strengthening yang. The results were shown in FIGS. 6 to 9, wherein C was the control group, M was the model group, G was the suncured ginseng group, R1, R2 and R3 were the processed product R1 group prepared in Example 1, the processed product R2 group prepared in Example 2, and the processed product R3 group prepared in Example 3, respectively.

### 2. Experiment Results

### 2.1 Comparison of the Organ Index

As can be seen from FIGS. 6 to 7, the model group showed a significantly decreased organ index; the administration group was improved as compared to the model group, but the difference was not significant. The reason for this phenomenon may be associated with the incompatibility or ineffective absorption caused by the intragastrical administration of drug powders to mice. Next, one may consider intragastrical administration of an aqueous solution of lyophilized powders after alcohol extraction and water extraction, which can effectively reduce the error due to indigestion or non-absorption in mice.

### 2.2. Comparison of the Total Plasma SOD Activity in Mice

As can be seen from FIG. 8, mice in the model group had a reduced plasma SOD activity when compared to the control group. After administration, mice in each group had an increased plasma SOD level, and the processed product groups R1 and R3 were significantly different from the model group (P <0.05), wherein the difference between the processed product group R3 and the model group was the most significant.

### 2.3 Comparison of Body Temperature of Mice Among Different Groups

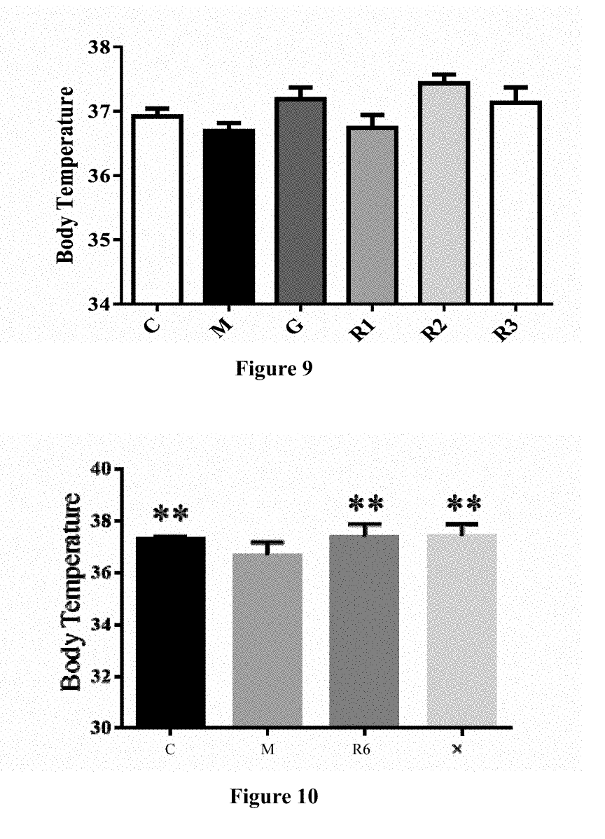

As can be seen from FIG. 9, mice in the control group had a body temperature of about 37° C. and the model group had a decreased body temperature. After administration, mice in each group generally had an increased body temperature, which was even above the normal level. Mice in the processed product group R2 and suncured ginseng groups had a body temperature above those in the normal group. Mice in the processed product group R1 had a body temperate close to those in the model group. The processed product group 3 showed a better effect.

### 3. Experiment Conclusion

In view of the above results, the R3 group had a significant difference from the model group (P <0.05).

The ginsenoside compositions prepared in Examples 4 to 6 were tested as above. The experiment results were similar to those for the ginsenoside compositions prepared in Examples 1 to 3, without any significant difference (P >0.05).

### EXAMPLE 9

### Effect of the Processing Process on the Activity of Strengthening Yang of Ginseng

### 1. Experiment Method

In the experiment of Example 8, since the administration was carried out by intragastrically administrating a water suspension of fine powders of the medicinal material, there might be a situation that the pharmaceutically active ingredients were not absorbed completely and the normal function of the digestive system of mice was affected, rendering their body weights and spleen functions impaired.

Since the spleen index is one of the experimental indexes, the inventors decided to extract fine powders of the medicinal materials (which were extracted twice with 75% alcohol, and each extraction was carried out for 4 h; after suction filtration, the dregs were extracted under hot reflux once with 200 ml of water; then the alcohol extract was evaporated to dryness under reduced pressure and mixed with the aqueous extract. The administration dosage of the aqueous extract was converted according to the original administration dosage. Thereafter, the aqueous extract was concentrated to the concentration for administration). After that, the extract of the medicinal materials was intragastrically administered.

A positive control group was set in this experiment, wherein the control medicinal material was seal pills (main ingredients: Radix Ginseng, Fructus Lycii, Pericarpium Zanthoxyli, Fructus Psoraleae, Ootheca Mantidis, Semen Juglandis, Rhizoma Ligustici Chuanxiong, Penis et Testis Cervi, Radix Achyranthis Bidentatae, Gecko, Fructus Rubi, Semen Cuscutae, Rhizoma Alismatis, Cortex Moutan Radicis, Penis Et Testis Canis, Testis Et Penis Canitis, Cortex Phellodendri, Cortex Cinnamomi, Radix Paeoniae Alba, Herba Epimedii, Rhizoma Dioscoreae, Cortex Eucommiae, Cornu Cervi Pantotrichum, Hippocampus, Radix Rehmanniae Preparata, Radix Angelicae Sinensis, Poria, Radix Morindae Officinalis, chrysotilum). This medicament warms kidney and activates yang, and is indicated for soreness and weakness of waist and knees, spiritlessness and lack of strength, cumbersome fatigued limbs, sensation of chill, frequent urination at night and shortness of breath and asthma due to deficiency of kidney yang. The dosage for mice was converted based on that for adults. As it is a pill, it was administrated after being pulverized, dissolved in CMC-Na, and sonicated for 10 min, which was prepared when it would be used.

Mice were divided into the following 4 groups, and each group comprised 12 animals.

(1) Control group (C): 0.4 ml pure water was intragastrically administered daily for 10 consecutive days.

(2) Model group (M): 0.5 mg hydrocortisone was intragastrically administered daily for 10 consecutive days.

(3) The processed ginseng product R6 group (R6) prepared in the present invention: the ginseng obtained from Example 3 was pulverized and passed through a 100-mesh screen to obtain fine powders, which were extracted twice with 75% alcohol, and each extraction was carried out for 4 h; after suction filtration, the dregs were extracted under hot reflux once with 200 ml of water; then the alcohol extract was evaporated to dryness under reduced pressure and mixed with the aqueous extract. The administration dosage of the aqueous extract was converted according to the original administration dosage. Thereafter, the aqueous extract was concentrated to the concentration for administration of 0.2 to 2 g/kg/d.

The intragastrical administration was initiated 3 days prior to start of the experiment and lasted for 13 consecutive days. The administration dosage of the medicinal material extract liquid was 0.65 g/kg/d by weight of the raw medicinal materials. Meanwhile, 0.5 mg/d of the molding agent hydrocortisone was administered for 10 consecutive days after 3 days of the administration.

(4) Positive medicament group (x): the above administration mode was used. The intragastrical administration was initiated 3 days prior to start of the experiment and lasted for 13 consecutive days. The administration dosage was 0.65 g/kg/d by weight of the pills. Meanwhile, 0.5 mg/d of the molding agent hydrocortisone was administered for 10 consecutive days after 3 days of the administration.

The modeling was performed for 10 days and the administration was initiated 3 days prior to modeling for pre-protection. On day 10 of modeling, the rectal temperature was measured. On day 11, the whole blood was retro-orbitally collected and centrifuged to harvest plasma. Upon dissection, the thymus and spleen indexes were measured, and then the plasma SOD and cAMP/cGMP levels were measured. They are listed in FIGS. 10 to 15, wherein C is the control group, M is the model group, R6 is the processed ginseng product R6 group prepared in Example 6, and x is the positive medicament group.

### 2. Experiment Results

### 2. Comparison of the Body Temperature of Mice Among Different Groups

On day 10, there was a significant difference in the body temperature of mice. The body temperature for the model group was lower, while those for the administration and positive groups were high and there was P<0.01 between them and the model group. The results were seen in FIG. 10.

### 2.2 Comparison of the Organ Index

In terms of the thymus index, R6 group was the best. The positive medicament and R6 groups were both improved as compared to the model group, and P<0.01.

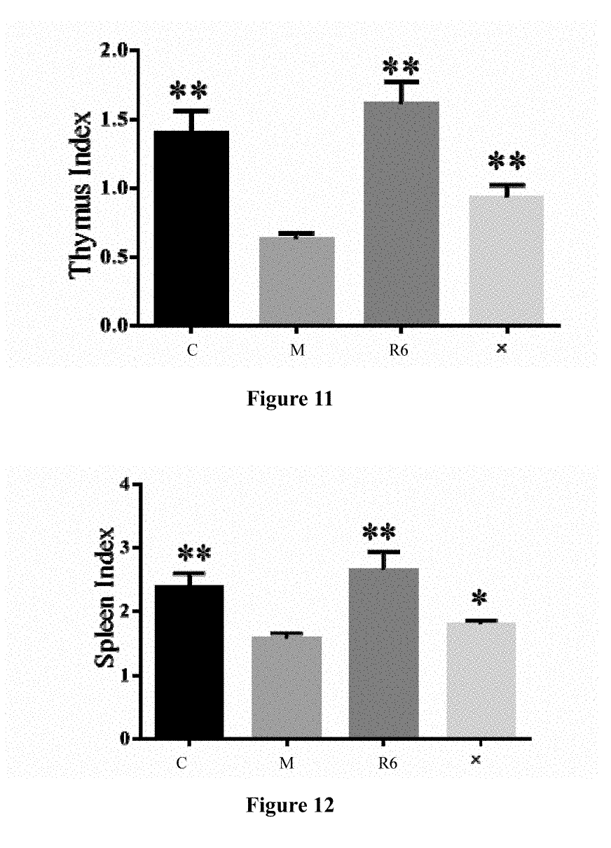

In terms of the spleen index, R6 group was the best and showed a significant difference. However, there was a relatively small difference between the positive medicament and the model groups, with P <0.05. Upon consideration, the reason for this phenomenon may be that what was administrated was not the aqueous extract of the pills, or the selected positive medicament had a poor efficacy. The experiment results were seen in FIGS. 11 to 12.

2.3 Plasma cAMP/cGMP Level in Mice

The cAMP/cGMP level is an important index evaluating the efficacy in a model with deficiency of kidney yang.

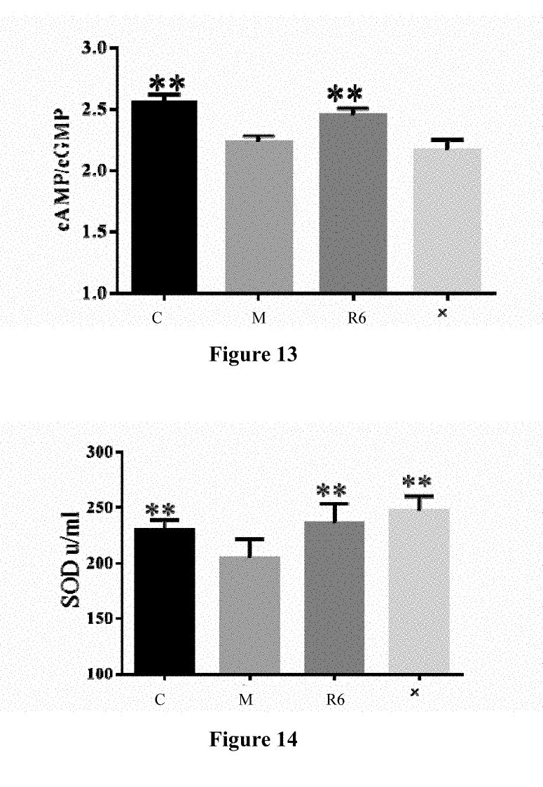

The positive medicament group was not significantly different from the model group, while there is P<0.01 between the administration and model groups. It was shown that, the aqueous extract of R6 indeed had a certain efficacy. The experiment results were seen in FIG. 13.

### 2.4 Total Plasma SOD Activity in Mice

The SOD results showed that the antioxidant ability of the body was improved in both the positive medicament and R3 groups. The experimental results were seen in FIG. 14.

### 2.5 Assay for the Plasma Testosterone Level in Mice

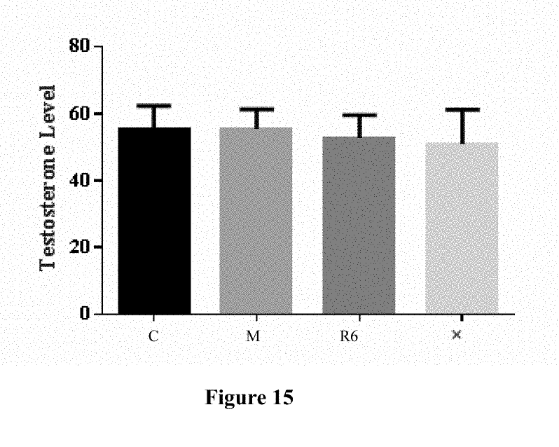

There was no obvious difference in the testosterone level in mice among the four groups of mice. This may be due to the low actual dosage of the modeling agent in this experiment, which is 0.5 fold of that in references. In addition, the intraperitoneal injection in the references was changed to intragastrical administration. As a result, the model group was modeled to a lesser extent. Furthermore, both the modeling agent and ginsenosides affect the function of sex gland indirectly, which may result in an insignificant difference in testosterone level. The particular experiment results were shown in FIG. 15.

### 3. Experiment Conclusions

In view of the above, the processed ginseng product R6 effectively improved the body temperature, thymus index and spleen index of the model mice with deficiency of kidney yang. In addition, as compared to the model group, the SOD activity was obviously enhanced and the plasma cAMP/cGMP level was also significantly increased. It was revealed that the processed ginseng product R6 improved the body function of mice with deficiency of kidney yang.

The ginsenoside compositions prepared in Examples 1-5 were tested as above. The experiment results were similar to those for the ginsenoside composition prepared in Example 6, without any significant difference (P>0.05).

The above description is merely preferred embodiments of the present invention. It should be noted that, several improvements and modifications could also be made by those of ordinary skill in the art without departing from the principle of the present invention, which should also be considered to be within the protection scope of the present invention.

1. A ginsenoside composition comprising rare ginsenosides Rh4, Rk3, Rk1 and Rg5.

2. The ginsenoside composition according to claim 1, further comprising ginsenosides Rf, Rb1, Rc, Rb2 and Rd.

3. The ginsenoside composition according to claim 1, further comprising ginsenosides Rg1 and Re.

4. The ginsenoside composition according to claim 3, wherein the total content of the rare ginsenosides Rh4, Rk3, Rk1 and Rg5 is greater than 42.55 mg/g; the total content of the ginsenosides Rf, Rb1, Rc, Rb2 and Rd is less than 10 mg/g; and the total content of the ginsenosides Rg1 and Re is less than 0.35 mg/g.

5. The ginsenoside composition according to claim 1, which is prepared by a method comprising steaming ginseng for 1 to 10 h at 90 to 130° C., wherein the steaming is carried out for 1 to 9 times.

6. The ginsenoside composition according to claim 5, wherein the method further comprises pulverizing the steamed ginseng and passing the pulverized ginseng through a 100-mesh screen.

7. The ginsenoside composition according to claim 5, wherein the method further comprises cutting the steamed ginseng into slices and extracting the slices with water or ethanol to obtain an extract.

8. A process for increasing thymus or spleen index, plasma SOD activity and/or plasma cAMP/cGMP level in a subject in need thereof, comprising administering the subject a medicament and/or healthcare product comprising the ginsenoside composition according to claim 1.

9. A process for treating deficiency of kidney yang in a subject in need thereof, comprising administering the subject a medicament and/or healthcare product comprising the ginsenoside composition according to claim 1.

10. The process according to claim 8, wherein the medicament is administrated at a dosage of 0.2 to 2 g/kg/d for a non-human animal subject, and at a human equivalent dosage of 0.02 to 0.2 g/kg/d for a human subject.

11. The process according to claim 9, wherein the medicament is administrated at a dosage of 0.2 to 2 g/kg/d for a non-human animal subject, and at a human equivalent dosage of 0.02 to 0.2 g/kg/d for a human subject.
